(19) United States
(12) Patent Application Publication
(10) Pub. No.: US 2017/0143890 A1
(43) Pub. Date: May 25, 2017

(54) Dual Lumen Retrograde Catheter and Hub Attachment

(71) Applicant: VascuTech Medical LLC, Oaks, PA (US)

(72) Inventor: Mahase Nardeo, Oaks, PA (US)

(73) Assignee: VascuTech Medical LLC, Oaks, PA (US)

(21) Appl. No.: 15/356,800

(22) Filed: Nov. 21, 2016

Related U.S. Application Data
(60) Provisional application No. 62/257,765, filed on Nov. 20, 2015.

(51) Int. Cl. A61M 1/36; A61M 25/01; A61M 25/00

(52) U.S. Cl. CPC A61M 1/3661; A61M 25/003; A61M 25/0097; A61M 25/0194; A61M 2025/0031

(57) ABSTRACT

Disclosed is a dual lumen retrograde catheter and hub attachment for dialysis. The catheter includes a venous lumen and an arterial lumen that are attached to a proximal hub having at least one luer extension extending therefrom. The distal end of the catheter is configured to be inserted into the blood vessel and placed into a desired position. The proximal end is tunneled backwards under the skin using a trocar and exited. The proximal end of the catheter can attach to a proximal hub via a pair of cannulas with raised barbs for removably securing proximal ends of the lumens thereon. The lumens are secured in a side-by-side configuration via a compression cap that can be screwed onto the proximal hub to prevent the catheter from detaching from the proximal hub.

### CROSS-REFERENCE TO RELATED APPLICATIONS

This application claims the benefit of U.S. Provisional Patent Application No. 62/257,765, filed Nov. 20, 2015, which is hereby incorporated by reference in its entirety.

### FIELD OF THE INVENTION

The present invention relates generally to a medical device. More particularly, the present invention is directed to a dual lumen retrograde catheter for a dialysis treatment and method of connecting the proximal end of the catheter to proximal molded tensions.

### BACKGROUND OF THE INVENTION

A dual lumen dialysis catheter generally comprises a distal end, a proximal end, and an integral longitudinal dual lumen tube that connects both of the ends to provide a conduit for moving blood or fluid to and from the body. These catheters are widely used in a routine dialysis procedure. The standard procedure typically requires a surgeon to tunnel the catheter under the skin and then secure the proximal hub in place before inserting the distal d into the blood vessel.

This standard procedure is disadvantageous, however, in that the distal end is rarely placed in an ideal position. Additionally, it is difficult to adjust the tip of the catheter once the hub is already anchored in place. With a conventional retrograde procedure, the distal end of the lumen is tunneled first and placed into position, and then the proximal end is tunneled backwards under the skin and exited over the clavicle.

While the backward tunneling improves the positioning of the distal end of the lumen, it does not allow for use of an integral proximal hub because it is impossible to maneuver the proximal end with a proximal hub integral thereto. In this regard, the invention described herein addresses this problem by providing a separate proximal hub and luer extensions that can removably attach to proximal ends of venous and arterial lumens to provide connections to blood lines once the proximal ends of the lumens exit the skin.

### SUMMARY OF THE INVENTION

In view of the disadvantages inherent in the known types of catheters and hub attachments now present in the prior art, the present invention provides an improved dual lumen catheter and hub attachment.

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as to prelude to the more detailed description that is disclosed later.

In some embodiments, the present invention comprises a dual lumen retrograde catheter comprising a first D-shaped cannula and a second D-shaped cannula, each of the cannulas having a proximal end and a distal end. The catheter comprises various distal configurations, depending upon the embodiment. In some embodiments, the distal end of the catheter comprises a short term step tip. In some embodiments, the distal end of the catheter comprises a retrograde long term step tip. In some embodiments, the distal end of the catheter comprises a retrograde long term split tip. In some embodiments, the distal end of the catheter comprises a long term split tip over wire implants. In some embodiments, the distal end of the catheter comprises a symmetrical split tip.

The proximal ends of the first and second cannula are joined via a compression cap. The compression cap is adapted to secure the proximal ends of the first and second cannula to a proximal hub when the proximal ends of the cannulas mate with respective barbed cannulas extending from the proximal hub. In one embodiment, the compression cap can be secured in place by mating threaded elements disposed on the interior wall thereof with complementary threaded elements disposed on the proximal hub.

The proximal hub establishes fluid communication between the dual lumen retrograde catheter and luer extensions. In some embodiments, the proximal hub is connected to a first luer extension and a second luer extension, each of the luer extensions connected to a female luer for blood line connection to provide a conduit for blood or fluid to and from the body. In some embodiments, each of the luer extensions further comprises a pinch clamp thereon.

In use, a distal end of the catheter is inserted into the blood vessel and placed into position, and then the proximal end of the catheter is tunneled backwards under the skin using a trocar and exited over the clavicle. Once the proximal end of the catheter exits the skin, the proximal hub with the barbs is attached to the lumens to provide connections to the bloodlines. A compression cap is then threaded over the barb connection site and screwed onto the proximal hub to prevent the catheter from easily detaching.

It is, therefore, an objective of the present invention to provide a new and improved hub attachment that can be used with a retrograde catheter having any distal configuration in a retrograde catheterization procedure.

It is another objective of the present invention to provide a connection of the proximal extension cannula to a catheter after backward tunneling is completed in a retrograde catheterization procedure.

It is still another objective of the present invention to provide a new and improved dual lumen retrograde catheter and hub attachment that increases the ease and convenience of a retrograde catheterization procedure.

It is still yet another objective of the present invention to provide a new and improved dual lumen retrograde catheter and hub attachment that improves the positioning of the distal end of the catheter during a retrograde catheterization procedure.

A final objective of the present invention to provide a new and improved dual lumen retrograde catheter and hub attachment that may be readily fabricated from materials that permit relative economy and commensurate with durability.

In the light of the foregoing, these and other objectives are accomplished in accordance with the principles of the present invention, wherein the novelty of the present invention will become apparent from the following detailed description and appended claims.

### BRIEF DESCRIPTION OF THE DRAWINGS

The above and other objects and advantages of the present invention will be apparent upon consideration of the following detailed description, taken in conjunction with the accompanying exemplary drawings, in which like reference characters refer to like parts throughout, and in which:

FIG. 1 shows a top elevational view of the proximal end of the present invention.

FIG. 2 shows a view of the distal end of a short term step tip catheter.

FIG. 3 shows a view of the distal end of a long term split tip.

FIG. 4 shows a view of the distal end of a symmetrical split tip.

### DETAILED DESCRIPTION OF THE INVENTION

The present invention is directed towards a catheter and hub attachment. For purposes of clarity, and not by way of limitation, illustrative views of the present invention are described with references made to the above-identified figures. Various modifications obvious to one skilled in the art are deemed to be within the spirit and scope of the present invention.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to disclose concepts in a concrete fashion. As used in this application, the term “or” is intended to mean an inclusive “or” rather than an exclusive “or.”

Additionally, the articles “a” and “an” as used in this application and the appended claims should generally be construed to mean “one or more” or “at least one” unless specified otherwise or clear from context to be directed to a singular form. Similarly, the terms “plurality” and “a plurality” as used herein includes, for example, “multiple” or “two or more.” For example, “a plurality of items” includes two or more items.

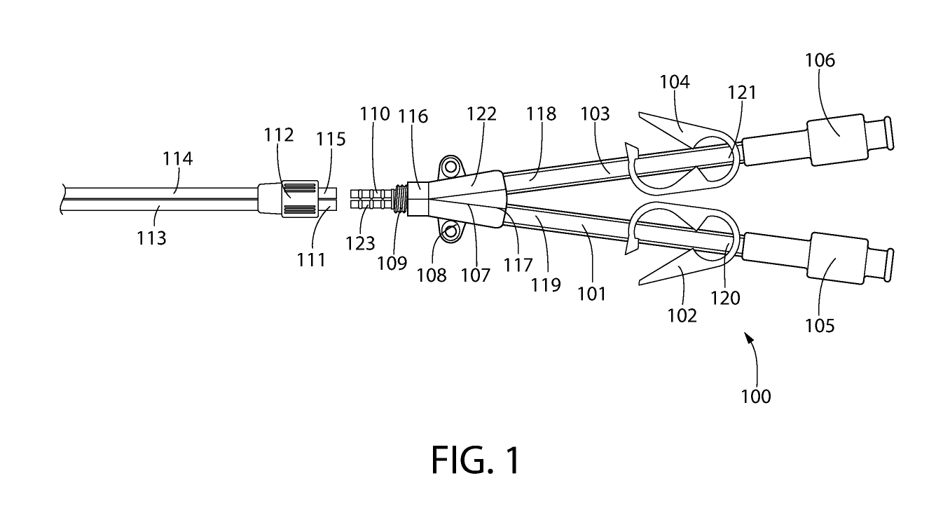

Referring now to FIG. 1, there is shown a view of the present invention in a disconnected configuration. The present invention comprises a dual lumen catheter and hub attachment 100. In some embodiments, the first lumen 114 comprises a venous lumen and the second lumen 113 comprises an arterial lumen. Preferably, the walls of the lumens 113, 114 comprise sufficient thickness in order to prevent kinking during storage, transport, and use.

Each of the venous lumen 114 and the arterial lumen 113 comprises a tube with a D-shape or a semi-circular cross section. In this way, the cross section of the venous lumen 114 and the arterial lumen 113 as joined together side-by-side form a substantially circular shape. Thus, the diameter of the cross sections of the venous lumen 114 and the arterial lumen 113 are substantially equal.

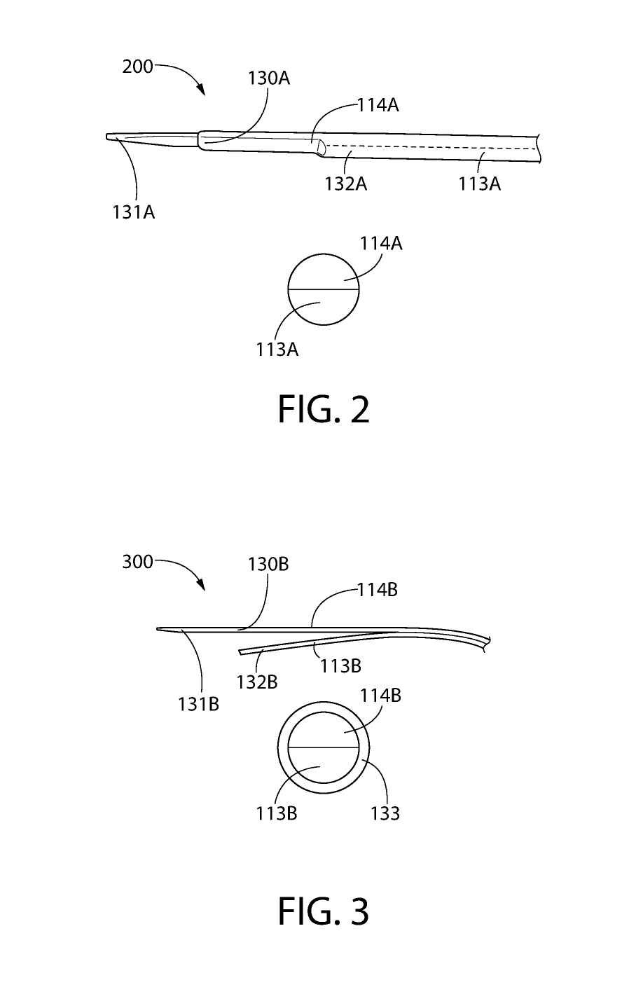

Each of the venous lumen 114 and the arterial lumen 113 comprises a proximal end 115, 111 and a distal end (exemplary embodiments shown in FIGS. 2 and 3), the distance between the proximal end 115, 111 and the distal end of each of the lumens 114, 113 defining a length of respective lumens 114, 113. In some embodiments, the length of the venous lumen 114 is greater than the length of the arterial lumen 113. The proximal ends 111, 115 of the lumens 113, 114 are aligned together and held in place via a compression cap 112. Thus, the distal end of the venous lumen 114 extends beyond the distal end of the arterial lumen 113. Alternatively, the length of the venous lumen 114 and the arterial lumen 113 are substantially equal. In this regard, the lumens need not be specifically designated as a venous lumen or an arterial lumen, and the connection can be reversed without high recirculation if one lumen is blocked.

The proximal ends 115, 111 of the lumens 113, 114 removably connect to a proximal hub 107. The proximal hub 107 comprises a first end 117 and a second end 116, the first end 117 comprising luer extensions 117, 122 extending therefrom, and the second end 116 comprising a first 110 and second cannula 123 with raised barbs extending therefrom. The cannulas 110, 123 comprise a substantially tubular structure. One terminal end of the cannulas 110, 123 is integral to the second end 116 of the proximal hub 107, and the opposing terminal end of the cannulas 110, 123 is free. In the illustrated embodiment, the proximal hub 107 tapers toward the second end 116 thereof.

Each of the first 110 and second cannulas 123 is in fluid communication with a first luer extension 103 and a second luer extension 101, respectively. In this regard, the proximal hub 107 comprises a pair of tunnels 122, each tunnel acting as a conduit for the cannulas 110, 123 and the luer extensions 103, 101. The tunnels 122 span the distance between the free terminal end of the cannulas 110, 123 to the first end 117 of the proximal hub 107. Additionally, the illustrated embodiment of the proximal hub 107 comprises suture holes 108 on opposing sides thereof.

Each of the first 110 and second cannula 123 is shaped and dimensioned to mate with the proximal ends 115, 111 of the venous lumen 114 and the arterial lumen 113, respectively. More specifically, the free terminal ends of the cannulas 110, 123 can be inserted into the proximal ends 115, 111 of the venous lumen 114 and the arterial lumen 113. Accordingly, the diameter of the cross section of the cannulas 110, 123 is less than the diameter of the cross section of the lumens 114, 113.

When the cannulas 110, 123 connect to the lumens 114, 113, the barbs facilitate in providing connections to bloodlines by preventing the lumens 114, 113 from easily or inadvertently separating from the cannulas 110, 123. It is contemplated that the barbs are configured to allow the lumens 114, 113 and the cannulas 110, 123 to connect relatively easily while improving separation force.

The proximal ends 115, 111 of the lumens 114, 113 are joined together via the compression cap 112. Optionally, the exterior of the compression cap comprises an identification cap for labeling. The interior of the compression cap 112 (i.e., near the end facing the proximal ends 115, 111) comprises threaded elements.

When the cannulas 110, 123 removably connect to the lumens 114, 113, the compression cap 112 can be placed over the connection site so that the threaded elements disposed within the interior of the compression cap 112 can engage complementary threaded elements 109 disposed on the second end 116 of the proximal hub 107. More specifically, the complementary threaded elements 109 are disposed near the base of the cannulas 110, 123. In this way, the compression cap 112 can removably attach to the proximal hub 107 in a screw-like manner and the compression cap 112 acts as a reinforcement feature for preventing the lumens 114, 113 from disengaging unintentionally.

Each of the first and second luer extensions 103, 101 comprises a distal end 118, 119 and a proximal end 121, 120. In some embodiments, the proximal hub 107 is molded to the proximal ends 118, 119 of the luer extensions 103, 101 so that it remains secured thereto and prevents leaking of fluids or other materials. Each of the proximal ends 121, 120 of the luer extensions 103, 101 comprises a female luer 106, 105 thereon for blood line connection. Additionally, the luer extensions 103, 101 comprise a clamp 104, 102 attached thereto. In the illustrated embodiment, the clamps 104, 102 comprise a pinch clamp.

In some embodiments, each clamp 104, 102 comprises a tag integral thereto. In this regard, each of the clamps can comprise a recessed portion for receiving the tag therein. The tag is shaped and dimensioned to fit snugly within the recessed portion. In this way, the tag is secured within the recessed portion via snap means or other fastening means. The outer surface of the tag comprises a printed label thereon. In some embodiments, the outer surface of the proximal hub may also comprise a printed label thereon.

Referring now to FIG. 2, there is shown a view of the distal end of a short term step tip catheter 200. The catheter comprises a venous lumen 114A and an arterial lumen 113A. The distal end 130A of the venous lumen 114A extend beyond the distal end 132A of the arterial lumen 113A. The distal end 130A of the venous lumen 114A comprises a pointed tip 131A. Each of the venous lumen 114A and the arterial lumen 113A comprises a D-shaped cross section so as to form a complete circular cross section when positioned together. The short term dialysis catheter can comprise D-shaped obturators in both lumens, depending upon the embodiment. The obturator can be configured to fit through the hub. It is contemplated that in other embodiments, the present invention may comprise a retrograde long term step tip, which is substantially equal to the short term step tip without an obturator.

Referring now to FIG. 3, there is shown a view of the distal end of a long term split tip 300. The catheter comprises a venous lumen 114B and an arterial lumen 113B. The distal end 130B of the venous lumen 114B extend beyond the distal end 132B of the arterial lumen 113B. The distal end 130B of the venous lumen 114B comprises a pointed tip 131B. The venous lumen 114B and the arterial lumen 113B are split at the distal ends 130B, 132B thereof. Each of the venous lumen 114B and the arterial lumen 113B comprises a D-shaped cross section so as to form a complete circular cross section when positioned together. In some embodiments, the venous lumen 114B and the arterial lumen 113B are held together within a sleeve 133 in order to accommodate lumens of different sizes (i.e., different cross section diameters).

In some embodiments, the venous lumen 114B may comprise a slit for receiving an obturator is inserted through, thereby allowing the obturator to transition from the arterial lumen 113B to the venous lumen 114B.

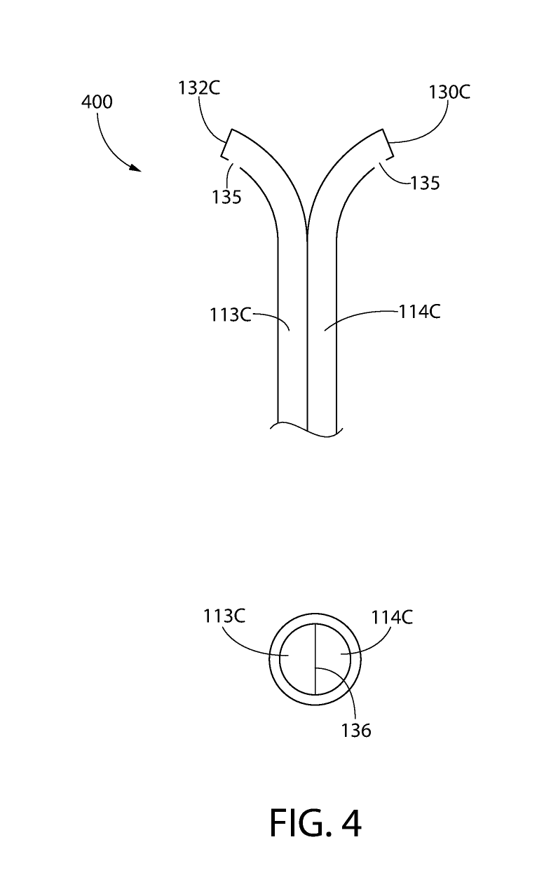

Referring now to FIG. 4, there is shown a distal end of a symmetrical split tip catheter 400. The catheter comprises a first lumen 114C and a second lumen 113C, wherein the two lumens are substantially symmetrical and are joined together and split at the distal ends 132C, 130C thereof. The cross section of the catheter 400 is substantially circular, and the lumens 114C, 113C are separated via a septum 136.

The distal ends 132C, 130C are bent apart from each other such that the catheter resembles a Y-shape when viewed from the front. Additionally, each of the distal ends 132C, 130C comprises a U-shaped slot 135 to prevent the inflow of blood from becoming blocked. Because the lumens 114C, 113C are not specifically designated as a venous lumen or an arterial lumen, the connection can be reversed without high recirculation if one of the lumens is blocked.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A retrograde catheter and hub attachment, comprising:
a dual lumen catheter comprising a first lumen and a second lumen secured together in a side-by-side configuration;
each of said first lumen and said second lumen comprising a distal end and a proximal end;
a proximal hub comprising a first end and a second end;
at least one luer extension extending from said first end of said proximal hub;
each of said proximal end of said first lumen and said proximal end of said second lumen removably connected to said second end of said proximal hub.

2. The catheter and hub attachment of claim 1, further comprising a clamp on said at least one luer.

3. The catheter and hub attachment of claim 1, further comprising:
a first cannula and a second cannula, wherein each of said first cannula and said second cannula extends from said second end of said proximal hub, further wherein said first cannula is configured to removably attach to said proximal end of said first lumen, and said second cannula is configured to removably attach to said proximal end of said second lumen.

4. The catheter and hub attachment of claim 3, wherein each of said first cannula and said second cannula comprises raised barbs along the length thereof, wherein said raised barbs are adapted to prevent said first lumen and said second lumen from easily disengaging said first cannula and said second cannula, respectively.

5. The catheter and hub attachment of claim 1, further comprising a compression cap configured to hold said first lumen and said second lumen together, wherein said compression cap is disposed near said proximal end of each of said first lumen and said second lumen, further wherein said compression cap is configured to removably attach onto said second end of said proximal hub to prevent said first lumen and said second lumen from detaching from said proximal hub.

6. The catheter and hub attachment of claim 1, wherein an interior wall of said compression cap comprises threaded elements thereon, further wherein said second end of said proximal hub comprises complementary threaded elements thereon, thereby allowing said threaded elements of said compression cap to engage said complementary threaded elements of said proximal hub.

7. The catheter and hub attachment of claim 1, wherein said proximal hub further comprises at least one suture hole.

8. The catheter and hub attachment of claim 1, wherein said at least one luer extension comprises a first luer extension and a second luer extension;
each of said first luer extension and said second luer extension comprising a female luer for establishing fluid connection.

9. The catheter and hub attachment of claim 1, wherein said first lumen comprises a venous lumen and said second lumen comprises an arterial lumen, further wherein a length of said first lumen is greater than a length of said second lumen.

10. The catheter and hub attachment of claim 1, wherein a length of said first lumen is substantially equal to a length of said second lumen such that said first lumen and said second lumen are symmetrical.

11. The catheter and hub attachment of claim 1, wherein said dual lumen catheter is a step tip catheter.

12. The catheter and hub attachment of claim 1, wherein said dual lumen catheter is a split tip catheter.

13. A method of providing connections to bloodlines, comprising the steps of:
inserting a distal end of a retrograde catheter into a blood vessel and placing said distal end into a desired position;
tunneling back a proximal end of said retrograde catheter under the skin;
exiting said proximal end of said retrograde catheter;
attaching a proximal hub to said proximal end of said retrograde catheter, wherein said proximal hub comprises at least one luer extension extending therefrom;
placing a compression cap over said proximal end of said retrograde catheter and screwing said compression cap onto said proximal hub to establish a fluid communication between said retrograde catheter and said at least one luer extension.

14. The method of claim 13, wherein said proximal hub further comprises a first end and a second end;
said first end of said proximal hub comprising said at least one luer extension extending therefrom, wherein said at least one luer extension is molded to said first end of said proximal hub;
said second end of said proximal hub connected to said proximal end of said retrograde catheter.

15. The method of claim 14, wherein said proximal hub further comprises at least one cannula, said at least one cannula extending from said second end of said proximal hub;
wherein said at least one cannula is configured to removably attach to said proximal end of said retrograde catheter.

16. The method of claim 14, wherein said retrograde catheter further comprises a compression cap, wherein said compression cap is disposed near said proximal end of said retrograde catheter, further wherein said compression cap is configured to removably attach onto said second end of said proximal hub to prevent said retrograde catheter from detaching from said proximal hub.
